(19) United States
(12) Patent Application Publication
(10) Pub. No.: US 2024/0389974 A1
(43) Pub. Date: Nov. 28, 2024

(54) ULTRASOUND DIAGNOSTIC APPARATUS

(71) Applicant: FUJIFILM Healthcare Corporation, Kashiwa-shi (JP)

(72) Inventors: Chizue Tanaka, Tokyo (JP); Hiroshi Kuribara, Chiba (JP)

(21) Appl. No.: 18/644,472

(22) Filed: Apr. 24, 2024

(30) Foreign Application Priority Data
May 26, 2023 (JP) 2023-086675

(51) Int. Cl. A61B 8/13; A61B 8/00; A61B 8/08

(52) U.S. Cl. CPC A61B 8/13; A61B 8/461; A61B 8/5207; A61B 8/5269

(57) ABSTRACT

For an ultrasound diagnostic apparatus, a learning model is trained to output an image adjustment parameter suitable for ultrasound data from input ultrasound data. A specific region decision unit specifies a specific region in a data space of the ultrasound data. A specific parameter decision unit inputs specific region data, which is ultrasound data of an inside of the specific region in the ultrasound data that is a processing target, to the trained learning model, and decides a specific parameter based on output of the learning model. An image quality adjustment processing unit executes image quality adjustment processing based on the specific parameter on the ultrasound data of the inside of the specific region, and executes image quality adjustment processing using a predetermined parameter for image quality adjustment different from the specific parameter on the ultrasound data of an outside of the specific region.

### BACKGROUND OF THE INVENTION

### 1. Field of the Invention

The present specification discloses an improvement of an ultrasound diagnostic apparatus.

### 2. Description of the Related Art

In the related art, there is known an ultrasound diagnostic apparatus that transmits and receives ultrasound waves to and from a subject, forms an ultrasound tomographic image based on a reception signal obtained by the transmission and reception of the ultrasound waves, and displays the formed ultrasound tomographic image (that is, a B-mode image) on a display. In addition, in the related art, a technique of enhancing an image quality of the ultrasound tomographic image in the ultrasound diagnostic apparatus is proposed.

For example, JP2020-114294A discloses an ultrasound diagnostic apparatus that can automatically apply appropriate image quality improvement processing based on a change in a signal amount of a subject. In the ultrasound diagnostic apparatus, in particular, it is attempted to improve a signal-to-noise ratio (S/N ratio) in accordance with a depth-dependent change in an echo signal from the subject. JP2020-503142A discloses an ultrasound imaging system that adjusts an ultrasound tomographic image formed from beam forming data by using output of a neural network trained to form the ultrasound tomographic image using channel data or the beam forming data from an ultrasound probe as input. JP2022-6869A discloses a medical image processing apparatus that cuts out different image regions from a plurality of medical images having different noise levels from each other, and combines the plurality of image regions to obtain a high-quality medical image.

### SUMMARY OF THE INVENTION

Various types of processing are executed on the reception signal until the ultrasound tomographic image is formed. By adjusting parameters related to these types of processing to appropriate values, a high-quality ultrasound tomographic image can be formed. Even after the ultrasound tomographic image is formed, the image quality enhancement processing may be executed on the ultrasound tomographic image. Also in this case, the high-quality ultrasound tomographic image can be formed by adjusting the parameters related to the image quality enhancement processing to appropriate values.

In the present specification, the processing on the reception signal for forming the ultrasound tomographic image and the image quality enhancement processing on the formed ultrasound tomographic image are collectively referred to as image quality adjustment processing. The parameters include a parameter related to processing on the reception signal for forming the ultrasound tomographic image and a parameter related to the image quality enhancement processing on the formed ultrasound tomographic image, and are referred to as image quality adjustment parameters.

Here, the contents of the appropriate image quality adjustment processing may be different for each region in the ultrasound tomographic image. In other words, the appropriate image quality adjustment parameters may be different for each region in the ultrasound tomographic image. Although the present invention is not limited to this, for example, since a high contrast is generally required for a region including a kidney in the ultrasound tomographic image, the appropriate image quality adjustment processing on the region is processing of realizing the high contrast, while since a high spatial resolution is generally required for a region including a liver in the ultrasound tomographic image, the appropriate image quality adjustment processing on the region is processing of realizing the high spatial resolution.

An object of the ultrasound diagnostic apparatus disclosed in the present specification is to provide an ultrasound diagnostic apparatus that can execute adaptive image quality adjustment processing on each region in an ultrasound tomographic image.

An aspect of the present specification discloses an ultrasound diagnostic apparatus comprising: a specific region decision unit that decides a specific region corresponding to a portion of an ultrasound tomographic image corresponding to ultrasound data obtained by transmitting and receiving ultrasound waves to and from a subject, in a data space of the ultrasound data; a specific parameter decision unit that decides a specific parameter, which is an image quality adjustment parameter for the specific region, based on output of a learning model in a case in which the ultrasound data of an inside of the specific region is input to the learning model that has been trained to output an image adjustment parameter suitable for the ultrasound data from the input ultrasound data by using learning data including the ultrasound data obtained by transmitting and receiving the ultrasound waves to and from the subject and a training image adjustment parameter to be used in image quality adjustment processing on the ultrasound data; and an image quality adjustment processing unit that executes image quality adjustment processing of adjusting an image quality of the inside of the specific region of the ultrasound tomographic image on the ultrasound data of the inside of the specific region based on the specific parameter, and executes image quality adjustment processing of adjusting an image quality of an outside of the specific region of the ultrasound tomographic image on the ultrasound data of the outside of the specific region by using a predetermined parameter for image quality adjustment different from the specific parameter.

With this configuration, the specific parameter suitable for the inside of the specific region is decided based on the output of the learning model to which the ultrasound data of the inside of the specific region is input. Then, the image quality adjustment processing can be executed on the ultrasound data of the inside of the specific region by using the specific parameter, and the image quality adjustment processing can be executed on the ultrasound data of the outside of the specific region by using the predetermined parameter. As a result, the adaptive image quality adjustment processing on each region in the ultrasound tomographic image is realized.

The image quality adjustment processing unit may use an image forming model in which the specific parameter is used as a weight parameter, to form the ultrasound tomographic image of the inside of the specific region, which has an adjusted image quality.

With this configuration, it is possible to form the ultrasound tomographic image of the inside of the specific region subjected to the image quality adjustment processing, by using the image forming model.

The image quality adjustment processing unit may execute image quality smoothing processing of smoothly changing an image quality at a boundary between the inside of the specific region and the outside of the specific region.

With this configuration, it is possible to smoothly change the image quality at the boundary between the inside of the specific region and the outside of the specific region on the ultrasound tomographic image.

The ultrasound diagnostic apparatus may further comprise: a display controller that causes a display unit to display a first ultrasound tomographic image in which the image quality adjustment processing based on the specific parameter is executed on the inside of the specific region and the image quality adjustment processing based on the predetermined parameter is executed on the outside of the specific region, and a second ultrasound tomographic image in which the image quality adjustment processing based on the predetermined parameter is executed on an entire region.

With this configuration, it is possible for the user to compare the first ultrasound tomographic image with the second ultrasound tomographic image.

The specific region decision unit may decide the specific region in accordance with an indication from a user of the ultrasound diagnostic apparatus.

With this configuration, it is possible for the user to execute the image quality adjustment processing on a desired region and the image quality adjustment processing on the other region as different types of processing.

The image quality adjustment processing unit may execute the image quality adjustment processing of adjusting the image quality of the inside of the specific region of the ultrasound tomographic image in accordance with an image quality adjustment policy corresponding to an indication from a user of the ultrasound diagnostic apparatus.

With this configuration, it is possible for the user to execute the image quality adjustment processing on the ultrasound data of the inside of the specific region with a desired image quality adjustment policy.

The specific parameter decision unit may store a combination of user identification information for identifying the user of the ultrasound diagnostic apparatus, a part of the subject included in the specific region decided in accordance with an indication from the user, and the image quality adjustment policy indicated by the user for the specific region, in a memory, and the image quality adjustment processing unit may execute image quality adjustment processing of adjusting the image quality of the inside of the specific region of the ultrasound tomographic image in accordance with the user identification information of the user and the image quality adjustment policy associated with the part in the memory in a case in which the specific region including the part is decided in accordance with the indication from the user.

With this configuration, it is possible for the user to execute the image quality adjustment processing of the ultrasound data of the inside of the specific region in accordance with the desired image adjustment policy without the need to indicate the image quality adjustment policy each time.

According to the ultrasound diagnostic apparatus disclosed in the aspect of the present specification, it is possible to provide the ultrasound diagnostic apparatus that can execute the adaptive image quality adjustment processing on each region in the ultrasound tomographic image.

### BRIEF DESCRIPTION OF THE DRAWINGS

FIG. 1 is a schematic configuration diagram of an ultrasound diagnostic apparatus according to the present embodiment.

FIG. 2 is a view showing a first example of a specific region decision screen.

FIG. 3 is a view showing a second example of the specific region decision screen.

FIG. 4 is a conceptual diagram showing decision processing of a specific parameter using a learning model.

FIG. 5 is a conceptual diagram showing processing of forming an ultrasound tomographic image using an image generative model based on the specific parameter.

FIG. 6 is a graph showing a weight of image quality adjustment processing at a boundary between an inside of a specific region and an outside of the specific region.

FIG. 7 is a diagram showing a display example of an ultrasound tomographic image in which the image quality adjustment processing based on the specific parameter is executed on the inside of the specific region, and an ultrasound tomographic image in which the image quality adjustment processing based on a predetermined parameter is executed on an entire region.

FIG. 8 is a view showing an example of an image quality adjustment policy decision screen.

### DESCRIPTION OF THE PREFERRED EMBODIMENTS

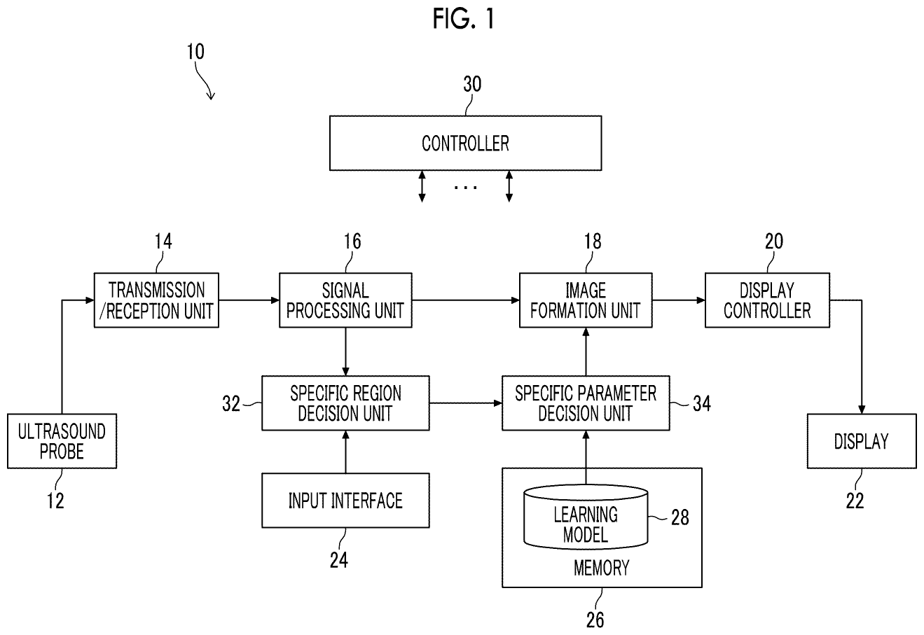

FIG. 1 is a schematic configuration diagram of an ultrasound diagnostic apparatus 10 according to the present embodiment. The ultrasound diagnostic apparatus 10 is a medical apparatus that is installed in a medical institution, such as a hospital, and is used during an ultrasound examination.

The ultrasound diagnostic apparatus 10 is an apparatus that scans a subject with an ultrasound beam to generate an ultrasound image based on a reception signal obtained by the scanning. In particular, in the present embodiment, the ultrasound diagnostic apparatus 10 forms an ultrasound tomographic image (B-mode image) in which an amplitude intensity of reflected waves from a scanning surface is transformed into brightness based on the reception signal. It should be noted that the ultrasound diagnostic apparatus 10 can also form other ultrasound images, such as a Doppler image representing a motion velocity of a tissue in the subject formed based on a difference (Doppler shift) in frequency between transmitted waves and received waves.

It should be noted that a transmission/reception unit 14, a signal processing unit 16, an image formation unit 18, a display controller 20, a specific region decision unit 32, and a specific parameter decision unit 34 provided in the ultrasound diagnostic apparatus 10 are configured by a processor. The processor includes at least one of a general-purpose processing apparatus (for example, a central processing unit (CPU)) or a dedicated processing apparatus (for example, a graphics processing unit (GPU), an application specific integrated circuit (ASIC), a field-programmable gate array (FPGA), a programmable logic device, and the like). The processor may be configured by the cooperation of a plurality of processing apparatuses that are present at physically separated positions, instead of being configured by one processing apparatus. In addition, each of the above-described units may be realized by a cooperation between hardware, such as the processor, and software.

An ultrasound probe 12 is a device that transmits and receives ultrasound waves to and from the subject. The ultrasound probe 12 has an oscillation element array including a plurality of oscillation elements that transmit and receive the ultrasound waves to and from the subject.

A transmission/reception unit 14 transmits a transmission signal to the ultrasound probe 12 (specifically, each oscillation element of the oscillation element array) under the control of a controller 30 (described later). As a result, the ultrasound waves are transmitted from each oscillation element toward the subject.

In addition, the transmission/reception unit 14 receives a reception signal from each oscillation element that receives the reflected waves from the subject. The transmission/reception unit 14 includes an adder and a plurality of delayers corresponding to the respective oscillation elements, and phase adjustment addition processing of aligning and adding phases of the reception signals from the respective oscillation elements is performed by the adder and the plurality of delayers. As a result, a reception beam signal in which information indicating the signal intensity of the reflected waves from the subject is arranged in a depth direction of the subject is formed. In the present specification, the reception signal output by each oscillation element before the phase adjustment addition processing is referred to as a channel signal, and the reception signal after the phase adjustment addition processing is referred to as the reception beam signal as described above. The reception signal before being transformed into data in a coordinate space of the ultrasound tomographic image by the image formation unit 18 described below may be collectively referred to as a radio frequency (RF) signal.

The reception beam signal before the signal processing via the signal processing unit 16 can be transitorily stored in a memory 26 or a memory (not shown) provided in the ultrasound diagnostic apparatus 10, for the image quality adjustment processing described below.

The signal processing unit 16 executes various types of signal processing including filter processing, such as applying a bandpass filter, and detection processing on the reception beam signal from the transmission/reception unit 14. Various types of signal processing in the signal processing unit 16 are processing that can affect an image quality of the ultrasound tomographic image formed by the image formation unit 18. That is, the signal processing unit 16 executes the image quality adjustment processing on the reception beam signal. The image quality adjustment processing executed by the signal processing unit 16 is executed based on an image quality adjustment parameter. The image quality adjustment parameter referred to by the signal processing unit 16 may be set by a user of the ultrasound diagnostic apparatus 10. In the present embodiment, the image quality adjustment parameter referred to by the signal processing unit 16 can be decided by the specific parameter decision unit 34 described below.

The image formation unit 18 forms the ultrasound tomographic image (B-mode image) based on the reception beam signal subjected to the signal processing via the signal processing unit 16. First, the image formation unit 18 transforms the reception beam signal into data in the coordinate space of the ultrasound image. In the present specification, the data after the transformation is referred to as a coordinate transformation signal. Then, the image formation unit 18 forms the ultrasound tomographic image based on the coordinate transformation signal.

In addition, the image formation unit 18 executes image quality enhancement processing, that is, image quality adjustment processing on the formed ultrasound tomographic image. The image quality adjustment processing executed by image formation unit 18 is also executed based on the image quality adjustment parameter. Although the image quality adjustment parameter referred to by the image formation unit 18 may also be set by the user of the ultrasound diagnostic apparatus 10, in the present embodiment, the image quality adjustment parameter referred to by the image formation unit 18 can also be decided by the specific parameter decision unit 34.

The ultrasound tomographic image before the image quality adjustment processing via the image formation unit 18 can also be transitorily stored in the memory 26 or the memory (not shown) provided in the ultrasound diagnostic apparatus 10, for the image quality adjustment processing described below.

A display controller 20 executes control of causing a display 22 to display the ultrasound tomographic image formed by the image formation unit 18 and various types of other information. The display 22 as a display unit is, for example, a display device configured by a liquid crystal display, an organic electro luminescence (EL), or the like.

An input interface 24 is configured by, for example, a button, a track ball, a touch panel, or the like. The input interface 24 is used to input a command from the user to the ultrasound diagnostic apparatus 10.

A memory 26 includes a hard disk drive (HDD), a solid state drive (SSD), an embedded multi media card (eMMC), a read only memory (ROM), or the like. The memory 26 stores an ultrasound diagnostic program for operating each of the units of the ultrasound diagnostic apparatus 10. It should be noted that the ultrasound diagnostic program can also be stored, for example, in a computer-readable non-transitory storage medium, such as a universal serial bus (USB) memory or a CD-ROM. The ultrasound diagnostic apparatus 10 can read and execute the ultrasound diagnostic program from such a storage medium.

As shown in FIG. 1, a learning model 28 is stored in the memory 26. The learning model 28 is, for example, a convolutional neural network (CNN) or the like, but may be any model as long as the functions described below are exhibited.

The learning model 28 is trained to output the image adjustment parameter suitable for the ultrasound data from the input ultrasound data by using learning data including the ultrasound data obtained by transmitting and receiving the ultrasound waves to and from the subject and the training image adjustment parameter to be used in the image quality adjustment processing on the ultrasound data. The ultrasound data included in the learning data of the learning model 28 includes the channel signal, the reception beam signal, the coordinate transformation signal, and the ultrasound tomographic image. In the present embodiment, the processing of training the learning model 28 is executed by a learning processing apparatus different from the ultrasound diagnostic apparatus 10, and the learning model 28, which has been sufficiently trained, is stored in the memory 26. However, the ultrasound diagnostic apparatus 10 may have a learning processing unit (not shown in FIG. 1), and the learning processing unit may execute the processing of training the learning model 28. In this case, the ultrasound diagnostic apparatus 10 is the learning processing apparatus.

Details of the processing of training the learning model 28 are as follows. The processor of the learning processing apparatus inputs the ultrasound data as the learning data to the learning model 28. The learning model 28 predicts the image quality adjustment parameter suitable for the input ultrasound data, and outputs the predicted image quality adjustment parameter. The processor of the learning processing apparatus changes the parameter in the learning model 28 such that a difference between the output data of the learning model 28 and the training image adjustment parameter included in the learning data is reduced. By repeating such processing, the learning model 28 is trained. The learning model 28, which has been sufficiently trained, can predict the image adjustment parameter suitable for the input ultrasound data with high accuracy.

The controller 30 includes at least one of a general-purpose processor (for example, a central processing unit (CPU)) or a dedicated processor (for example, a graphics processing unit (GPU), an application specific integrated circuit (ASIC), a field-programmable gate array (FPGA), a programmable logic device, and the like). The controller 30 may be configured by the cooperation of a plurality of processing apparatuses that are present at physically separated positions, instead of being configured by one processing apparatus. The controller 30 controls each of the units of the ultrasound diagnostic apparatus 10 in accordance with the ultrasound diagnostic program stored in the memory 26.

The specific region decision unit 32 specifies a specific region corresponding to a portion of the ultrasound tomographic image (in other words, the ultrasound tomographic image formed based on the ultrasound data, or the ultrasound data itself in a case in which the ultrasound data is the ultrasound tomographic image) corresponding to the ultrasound data in the data space of the ultrasound data, which is obtained by transmitting and receiving the ultrasound waves to and from the subject, that is, the channel signal, the reception beam signal, the coordinate transformation signal, or the ultrasound tomographic image. It should be noted that FIG. 1 shows that the specific region decision unit 32 receives the ultrasound data (that is, the reception beam signal) from the signal processing unit 16, but as described above, the ultrasound data may be various signals, so that the specific region decision unit 32 may receive the channel signal as the ultrasound data from the transmission/reception unit 14 or may receive the coordinate transformation signal or the ultrasound tomographic image as the ultrasound data from the image formation unit 18.

As a first method of the method of specifying the specific region, the specific region decision unit 32 can specify the specific region by analyzing the ultrasound data (here, particularly, the reception beam signal, the coordinate transformation signal, or the ultrasound tomographic image).

For example, a region specification learning model that is trained by using ultrasound data and learning data including a position and a name of a part (an organ or the like) included in the ultrasound data is prepared in advance, the region specification learning model being trained to predict the position and the name of the part included in the ultrasound data based on the input ultrasound data, and output the predicted position and name. Then, the specific region decision unit 32 can specify the specific region based on the output of the trained region specification learning model in a case in which the ultrasound data that is the processing target is input.

In addition, for example, partial ultrasound data indicating a desired specific region can be prepared in advance as template data, a degree of similarity between each portion of the ultrasound data that is the processing target and the template data can be calculated, and a region in which the degree of similarity is equal to or greater than a threshold value and is the maximum can be specified as the specific region.

As a second method of the method of specifying the specific region, the specific region decision unit 32 can specify the specific region in accordance with an indication from the user of the ultrasound diagnostic apparatus 10.

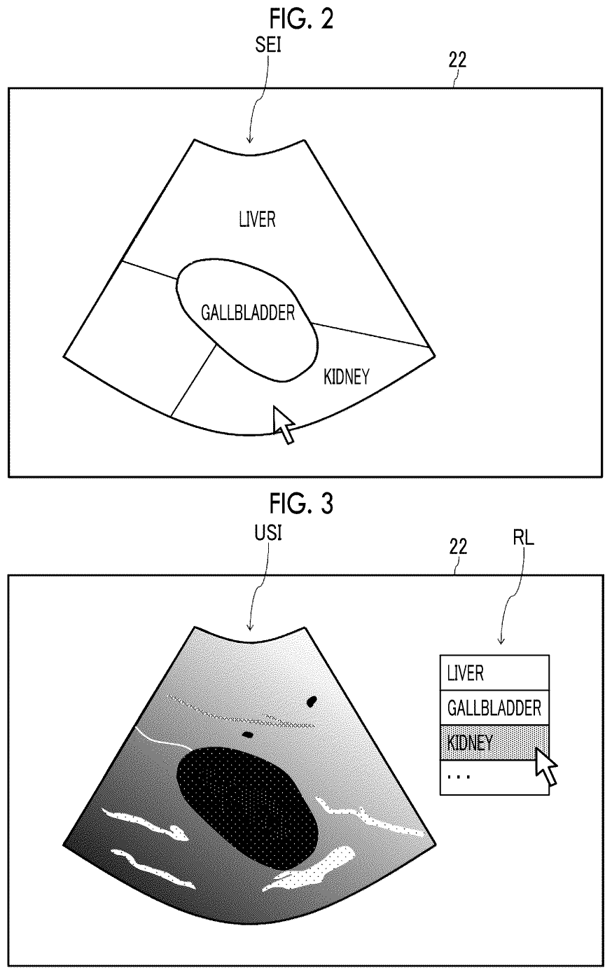

For example, the image formation unit 18 forms a segmentation image in which the ultrasound tomographic image is divided into a plurality of regions (for example, corresponding to the respective organs) by using the existing technology such as using the learning model, and the display controller 20 causes the display 22 to display the formed segmentation image SEI as shown in FIG. 2. In the example of FIG. 2, the segmentation image SEI including a liver region, a gallbladder region, and a kidney region is shown. Then, the user selects a desired region from the displayed segmentation image SEI with a cursor. The specific region decision unit 32 specifies a region of the segmentation image SEI selected by the user, as the specific region.

The image formation unit 18 specifies one or a plurality of region names (for example, organ names) included in the ultrasound tomographic image by using the existing technology such as using the learning model, and the display controller 20 causes the display 22 to display a region name list RL, which is a list of the specified region names, together with an ultrasound tomographic image USI as shown in FIG. 3. Then, the user selects a desired region from the displayed region name list RL with a cursor. The specific region decision unit 32 specifies the region selected by the user, as the specific region.

The method of specifying the specific region in accordance with the indication from the user is not limited to the above method. For example, the user may directly designate the specific region by using the cursor or the like on the ultrasound tomographic image displayed on the display 22.

Returning to FIG. 1, the specific parameter decision unit 34 uses the trained learning model 28 to decide the specific parameter, which is the image quality adjustment parameter for the specific region specified by the specific region decision unit 32.

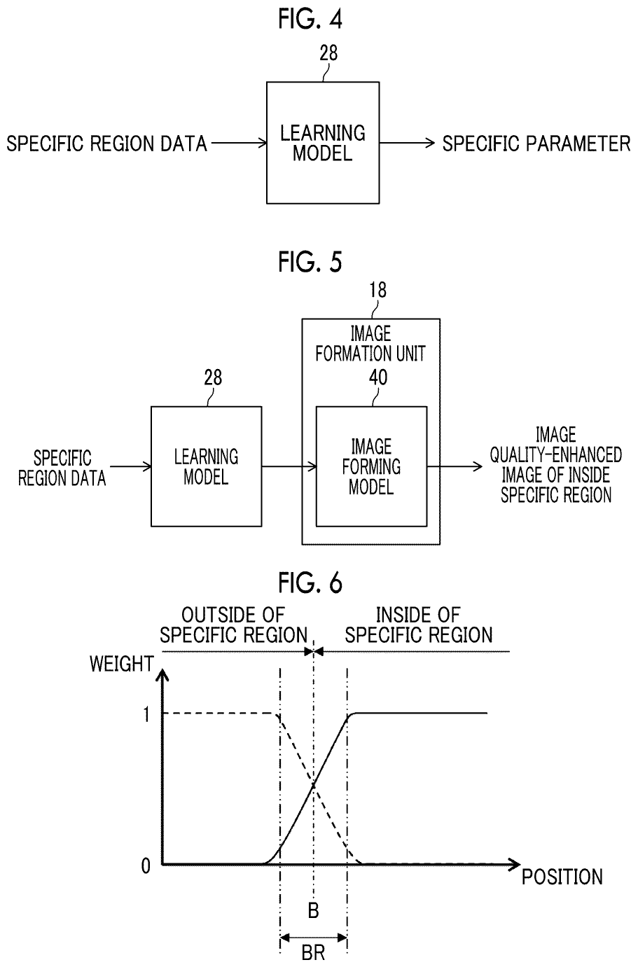

Specifically, the specific parameter decision unit 34 cuts out specific region data, which is the ultrasound data of the inside of the specific region, from the ultrasound data that is the processing target (that is, the channel signal, the reception beam signal, the coordinate transformation signal, or the ultrasound tomographic image). As shown in FIG. 4, the specific parameter decision unit 34 inputs the specific region data to the trained learning model 28. The learning model 28 predicts the image quality adjustment parameter suitable for the input specific region data, and outputs the predicted image quality adjustment parameter. The specific parameter decision unit 34 decides the specific parameter based on the output of the learning model 28.

Here, it should be noted that the specific parameter decision unit 34 inputs only the specific region data, which is a part of the ultrasound data, to the learning model 28, instead of the entire ultrasound data. As a result, the learning model 28 predicts the image quality adjustment parameter specialized for the specific region data, instead of the entire ultrasound data, and outputs the predicted image quality adjustment parameter. That is, it can be said that the specific parameter is an image quality adjustment parameter specialized for the specific region.

For example, in a case in which the specific region is the kidney region, the kidney region requires a high contrast, and thus a parameter that realizes the reduction of a dynamic range or the filter processing with an emphasis on the high contrast is decided as the specific parameter. In addition, for example, in a case in which the specific region is the liver region, since the liver region requires substantial smooth visualization, a time gain compensation (TGC) in which the brightness is uniform from a shallow part to a deep part or a parameter that realizes filter processing with an emphasis on a high spatial resolution is decided as the specific parameter. In addition, for example, in a case in which the specific region is the gallbladder region, it is required in the gallbladder region that an abnormality such as a polyp can be detected, and thus a parameter that realizes the filter processing of reducing an artifact that inhibits the discovery of the abnormality, such as a multiple reflection artifact, is decided as the specific parameter. Further, for example, in a case in which the specific region is a breast region, the breast region requires a diagnosis of a mammary gland region, and thus a parameter is decided in which only the mammary gland region to be diagnosed is a high-definition image and the brightness of the other regions is lowered so as not to hinder the diagnosis, as the specific parameter.

The signal processing unit 16 or the image formation unit 18 as an image quality adjustment processing unit executes the image quality adjustment processing on the ultrasound data of the inside of the specific region based on the specific parameter decided by the specific parameter decision unit 34.

First, the image quality adjustment processing via the signal processing unit 16 will be described. The signal processing unit 16 executes the image quality adjustment processing on the stored reception beam data before the signal processing based on the specific parameter decided by the specific parameter decision unit 34. Specifically, the signal processing unit 16 executes the image quality adjustment processing on the reception beam signal of the inside of the specific region based on the specific parameter, and executes the image quality adjustment processing on the reception beam signal of the outside of the specific region by using the predetermined parameter for image quality adjustment different from the specific parameter. The predetermined parameter for image quality adjustment may be, for example, a parameter set in advance by the user or a predetermined parameter in the ultrasound diagnostic apparatus 10. As described above, the signal processing unit 16 can serve as the image quality adjustment processing unit.

Next, the image quality adjustment processing via the image formation unit 18 will be described. The image formation unit 18 executes the image quality adjustment processing on the stored ultrasound tomographic image before the image quality adjustment processing based on the specific parameter decided by the specific parameter decision unit 34. Specifically, the image formation unit 18 executes the image quality adjustment processing on the ultrasound tomographic image of the inside of the specific region based on the specific parameter, and executes the image quality adjustment processing on the ultrasound tomographic image of the outside of the specific region by using the predetermined parameter for image quality adjustment different from the specific parameter. Here as well, the predetermined parameter for image quality adjustment may be, for example, a parameter set in advance by the user or a predetermined parameter in the ultrasound diagnostic apparatus 10. As described above, the image formation unit 18 can also serve as the image quality adjustment processing unit.

As described above, the signal processing unit 16 or the image formation unit 18 executes different types of the image quality adjustment processing on the ultrasound data of the inside of the specific region and the ultrasound data of the outside of the specific region based on the image quality adjustment parameters different from each other. As a result, it is possible to execute the adaptive image quality adjustment processing on each region in the ultrasound tomographic image. For example, the image quality adjustment processing can be executed on the kidney region, which is the specific region such that the image quality is adjusted to have a high contrast, and the image quality adjustment processing can be executed on the other regions such that the contrast is not excessively high and is moderate.

In a case in which the specific region decision unit 32 decides the specific region in accordance with the indication from the user, the user can execute the image quality adjustment processing on the desired region and the image quality adjustment processing on the other region as different types of processing.

In particular, in the present embodiment, for example, the image quality adjustment processing can be executed by using different specific parameters for each ultrasound data even in a case of the region corresponding to the same organ. For example, it is assumed that the liver region is decided as a first specific region in first ultrasound data, and the same liver region is decided as a second specific region in second ultrasound data different from the first ultrasound data. In this case, in a case in which the contents of the first ultrasound data of the inside of the first specific region and the second ultrasound data of the inside of the second specific region are different from each other, the learning model 28 can output different image quality adjustment parameters between a case in which the first ultrasound data of the inside of the first specific region is input and a case in which the second ultrasound data of the inside of the second specific region is input. As a result, according to the present embodiment, it is possible to execute different types of the image quality adjustment processing depending on the ultrasound data for the same liver region.

The specific parameter decision unit 34 inputs only the specific region data in the ultrasound data to the learning model 28, instead of the entire ultrasound data. As a result, it is possible to reduce an amount of calculation for the learning model 28 to predict the specific parameter output the predicted specific parameter.

In the present embodiment, the image formation unit 18 forms the ultrasound tomographic image based on the reception beam data for the inside of the specific region, and then executes the image quality adjustment processing of the ultrasound tomographic image based on the specific parameter. However, as shown in FIG. 5, the image formation unit 18 as the image quality adjustment processing unit may form the ultrasound tomographic image of the inside of the specific region, which has an adjusted image quality, by using an image forming model 40 in which the specific parameter is a weight parameter. The image forming model 40 may be, for example, a learning model, such as a generative adversarial network (GAN). In this case, the learning model 28 may be trained in advance such that a format of the output data is a format suitable for the image forming model 40.

In the present embodiment, the image formation unit 18 executes the image quality adjustment processing of the ultrasound tomographic image of the inside of the specific region based on the specific parameter or the formation processing of the ultrasound tomographic image of the inside of the specific region using the image forming model 40, but these processing may be executed by the specific parameter decision unit 34.

In the present embodiment, different types of the image quality adjustment processing are executed between the inside of the specific region and the outside of the specific region. Therefore, the difference in image quality may be remarkable at a boundary between the inside of the specific region and the outside of the specific region. Therefore, the signal processing unit 16 or the image formation unit 18 as the image quality adjustment processing unit may execute the image quality smoothing processing of smoothly changing the image quality at the boundary between the inside of the specific region and the outside of the specific region.

FIG. 6 is a graph showing a weight of the image quality adjustment processing at the boundary between the inside of the specific region and the outside of the specific region. In FIG. 6, a boundary B between the inside of the specific region and the outside of the specific region is indicated by a one-point chain line, and a left side of the boundary B is the outside of the specific region and a right side of the boundary B is the inside of the specific region. A certain region in a vicinity of the boundary B between the outside of the specific region and the inside of the specific region is referred to as a boundary region BR. A solid line graph represents the weight of the image quality adjustment processing using the specific parameter on the inside of the specific region, and a broken line graph represents the weight of the image quality adjustment processing using the predetermined parameter on the outside of the specific region.

As shown in FIG. 6, the inside of the specific region other than the boundary region BR, the weight of the image quality adjustment processing using the specific parameter is “1”, and the weight of the image quality adjustment processing using the predetermined parameter is “0”. This means that the image quality adjustment processing using only the specific parameter is executed on the ultrasound data of the inside of the specific region other than the boundary region BR. In the boundary region BR, the weight of the image quality adjustment processing using the specific parameter is gradually decreased from the inside of the specific region to the outside of the specific region (from the right to the left in the graph of FIG. 6), and the weight of the image quality adjustment processing using the predetermined parameter is increased. This means that, in the boundary region BR, a result of the image quality adjustment processing using the specific parameter and a result of the image quality adjustment processing using the predetermined parameter are combined in accordance with the respective weights. In the present embodiment, the processing corresponds to the image quality smoothing processing. In the outside of the specific region other than the boundary region BR, the weight of the image quality adjustment processing using the specific parameter is “0”, and the weight of the image quality adjustment processing using the predetermined parameter is “1”. This means that the image quality adjustment processing using only the specific parameter is executed on the ultrasound data of the inside of the specific region other than the boundary region BR.

In this way, in the boundary region BR, the result of the image quality adjustment processing using the specific parameter and the result of the image quality adjustment processing using the predetermined parameter are combined, so that the image quality can be smoothly changed at the boundary between the inside of the specific region and the outside of the specific region on the ultrasound tomographic image.

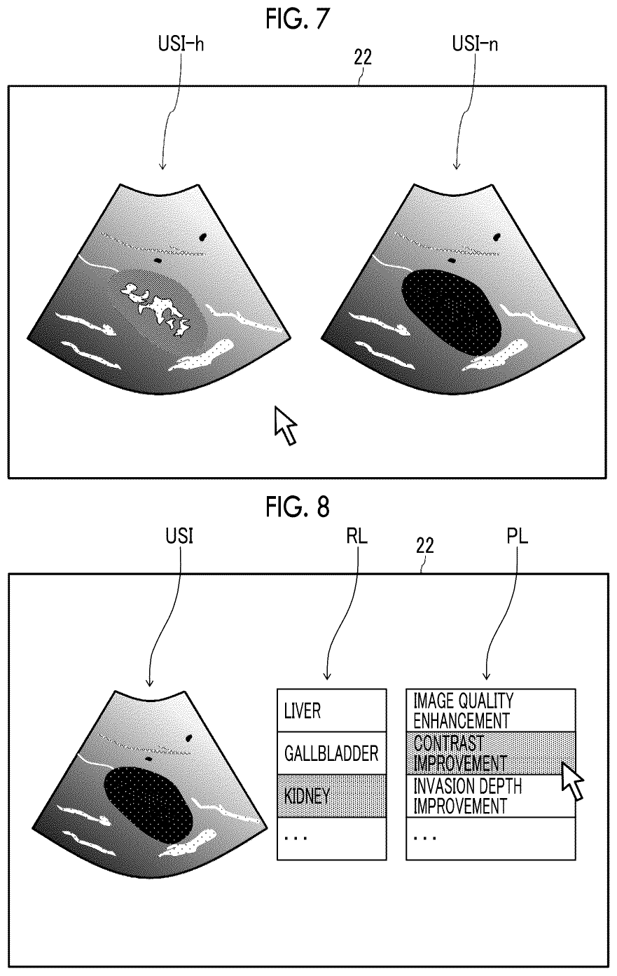

The display controller 20 causes the display 22 to display the ultrasound tomographic image on which the image quality adjustment processing is executed. As shown in FIG. 7, the display controller 20 may causes the display 22 to display a first ultrasound tomographic image USI-h in which the image quality adjustment processing based on the specific parameter is executed on the inside of the specific region and the image quality adjustment processing based on the predetermined parameter is executed in the outside of the specific region is executed, and a second ultrasound tomographic image USI-n in which the image quality adjustment processing based on the predetermined parameter is executed on the entire region. As a result, it is possible for the user to compare the first ultrasound tomographic image USI-h with the second ultrasound tomographic image USI-n.

In order to display the second ultrasound tomographic image USI-n on the display 22, the signal processing unit 16 executes the image quality adjustment processing using the predetermined parameter on an entire reception frame signal for one frame, and the image formation unit 18 executes the image quality adjustment processing using the predetermined parameter on the entire formed ultrasound tomographic image. As a result, the second ultrasound tomographic image USI-n is formed.

As described above, appropriate image quality adjustment processing may be different for each region of the ultrasound tomographic image, but there may be a case in which a plurality of types of appropriate image quality adjustment processing are present for one region. For example, in a case in which the specific region is the kidney region, as the appropriate image quality adjustment processing on the kidney region, there is processing of increasing a resolution, processing of improving a contrast, processing of increasing an invasion depth, and the like. These types of processing are types of processing executed by using different image adjustment parameters (that is, the specific parameters).

Therefore, the signal processing unit 16 or the image formation unit 18 as the image quality adjustment processing unit may execute the image quality adjustment processing of adjusting the image quality of the inside of the specific region of the ultrasound tomographic image in accordance with the image quality adjustment policy corresponding to the indication from the user of the ultrasound diagnostic apparatus 10.

As a premise, the learning model 28 is trained to output the image adjustment parameter suitable for the ultrasound data and in accordance with the image quality adjustment policy from the input ultrasound data and the image quality adjustment policy by using learning data including the ultrasound data, the image quality adjustment policy, and the training image adjustment parameter to be used in the image quality adjustment processing in accordance with the image quality adjustment policy for the ultrasound data. This configuration may be realized by configuring one learning model 28 to be able to receive the image quality adjustment policy, or may be realized by training different learning models 28 for each image quality adjustment policy.

FIG. 8 is a diagram showing an example of an image quality adjustment policy decision screen. In the example of FIG. 8, the user selects the kidney region as the specific region from the region name list RL described above. Further, the display controller 20 causes the display 22 to display a policy list PL in which a plurality of image quality adjustment policies of the image quality adjustment processing suitable for the selected specific region (kidney region in the example of FIG. 8). In a case in which the user selects a desired image adjustment policy from the policy list PL, the specific parameter decision unit 34 inputs the specific region data corresponding to the kidney region, which is the specific region, and the image adjustment policy selected by the user to the learning model 28. The learning model 28 predicts the image quality adjustment parameter suitable for the input specific region data and in accordance with the input image adjustment policy, and outputs the predicted image quality adjustment parameter. The specific parameter decision unit 34 decides the specific parameter based on the output of the learning model 28. Thereafter, the signal processing unit 16 or the image formation unit 18 executes the image quality adjustment processing on the ultrasound data of the inside of the specific region based on the specific parameter.

It is considered that the same user often executes the image quality adjustment processing with the same image adjustment policy on the specific region including the same part of the subject. Therefore, in the present embodiment, the signal processing unit 16 or the image formation unit 18 as the image quality adjustment processing unit may store the image adjustment policy selected for the specific region including the part by a certain user, and then may decide, in a case in which the specific region including the part is decided in accordance with the indication of the user, the image adjustment policy for a newly decided specific region based on the image adjustment policy selected for the specific region including the part by the user in the past.

Specifically, each time the user selects the image adjustment policy on the image quality adjustment policy decision screen shown in FIG. 8, the specific parameter decision unit 34 accumulates and stores, in a policy database (not shown in FIG. 1) constructed in the memory 26, a combination of a user ID as user identification information for uniquely identifying the user, a part (in the example of FIG. 8, “kidney”) of the subject included in the specific region decided in accordance with the indication from the user, and the image quality adjustment policy (in the example of FIG. 8, “contrast improvement”) indicated by the user for the specific region. It should be noted that the user ID of the user can be specified by the controller 30 in a case in which the user starts to use the ultrasound diagnostic apparatus 10 by authenticating the user.

Then, for example, in a case in which the specific parameter decision unit 34 decides the specific region including the part (in this example, “kidney”) in accordance with the indication of the user for other ultrasound data, the specific parameter decision unit 34 specifies the user ID of the user and the image adjustment policy associated with the part, with reference to the policy database. In the present example, “contrast improvement” is specified. Thereafter, the specific parameter decision unit 34 inputs the specific region data corresponding to the kidney region, which is the specific region, and the specified image adjustment policy to the learning model 28, and decides the specific parameter based on the output of the learning model 28. It is needless to say that the specific parameter is an image adjustment parameter in accordance with the specified image adjustment policy. Thereafter, the signal processing unit 16 or the image formation unit 18 executes the image quality adjustment processing on the ultrasound data of the inside of the specific region based on the specific parameter (in other words, in accordance with the specified image adjustment policy).

As a result, the user can execute the image quality adjustment processing of the ultrasound data of the inside of the specific region in accordance with the desired image adjustment policy without the need to indicate the image quality adjustment policy each time.

Although the embodiment according to the present invention has been described above, the present invention is not limited to the embodiment described above, and various modifications can be made without departing from the gist of the present invention.

The present application claims priority from Japanese Patent Application No. 2023-086675 filed on May 26, 2023, the content of which is hereby incorporated by reference into this application.

### EXPLANATION OF REFERENCES

10: ultrasound diagnostic apparatus
12: ultrasound probe
14: transmission/reception unit
16: signal processing unit
18: image formation unit
20: display controller
22: display
24: input interface
26: memory
28: learning model
30: controller
32: specific region decision unit
34: specific parameter decision unit
40: image forming model

What is claimed is:

1. An ultrasound diagnostic apparatus comprising:
a specific region decision unit that decides a specific region corresponding to a portion of an ultrasound tomographic image corresponding to ultrasound data obtained by transmitting and receiving ultrasound waves to and from a subject, in a data space of the ultrasound data;
a specific parameter decision unit that decides a specific parameter, which is an image quality adjustment parameter for the specific region, based on output of a learning model in a case in which the ultrasound data of an inside of the specific region is input to the learning model that has been trained to output an image adjustment parameter suitable for the ultrasound data from the input ultrasound data by using learning data including the ultrasound data obtained by transmitting and receiving the ultrasound waves to and from the subject and a training image adjustment parameter to be used in image quality adjustment processing on the ultrasound data; and
an image quality adjustment processing unit that executes image quality adjustment processing of adjusting an image quality of the inside of the specific region of the ultrasound tomographic image on the ultrasound data of the inside of the specific region based on the specific parameter, and executes image quality adjustment processing of adjusting an image quality of an outside of the specific region of the ultrasound tomographic image on the ultrasound data of the outside of the specific region by using a predetermined parameter for image quality adjustment different from the specific parameter.

2. The ultrasound diagnostic apparatus according to claim 1,
wherein the image quality adjustment processing unit uses an image forming model in which the specific parameter is used as a weight parameter, to form the ultrasound tomographic image of the inside of the specific region, which has an adjusted image quality.

3. The ultrasound diagnostic apparatus according to claim 1,
wherein the image quality adjustment processing unit executes image quality smoothing processing of smoothly changing an image quality at a boundary between the inside of the specific region and the outside of the specific region.

4. The ultrasound diagnostic apparatus according to claim 1, further comprising:
a display controller that causes a display unit to display a first ultrasound tomographic image in which the image quality adjustment processing based on the specific parameter is executed on the inside of the specific region and the image quality adjustment processing based on the predetermined parameter is executed on the outside of the specific region, and a second ultrasound tomographic image in which the image quality adjustment processing based on the predetermined parameter is executed on an entire region.

5. The ultrasound diagnostic apparatus according to claim 1,
wherein the specific region decision unit decides the specific region in accordance with an indication from a user of the ultrasound diagnostic apparatus.

6. The ultrasound diagnostic apparatus according to claim 1,
wherein the image quality adjustment processing unit executes the image quality adjustment processing of adjusting the image quality of the inside of the specific region of the ultrasound tomographic image in accordance with an image quality adjustment policy corresponding to an indication from a user of the ultrasound diagnostic apparatus.

7. The ultrasound diagnostic apparatus according to claim 6,
wherein the specific parameter decision unit stores a combination of user identification information for identifying the user of the ultrasound diagnostic apparatus, a part of the subject included in the specific region decided in accordance with an indication from the user, and the image quality adjustment policy indicated by the user for the specific region, in a memory, and
the image quality adjustment processing unit executes image quality adjustment processing of adjusting the image quality of the inside of the specific region of the ultrasound tomographic image in accordance with the user identification information of the user and the image quality adjustment policy associated with the part in the memory in a case in which the specific region including the part is decided in accordance with the indication from the user.
